(19) United States
(12) Patent Application Publication
(10) Pub. No.: US 2026/0053436 A1
(43) Pub. Date: Feb. 26, 2026

(54) DIAGNOSING AND TREATING ATTENTION DEFICIT HYPERACTIVITY DISORDER

(71) Applicant: Yeda Research and Development Co. Ltd., Rehovot (IL)

(72) Inventors: Noam SOBEL, Jaffa (IL); Timna SOROKA, Rehovot (IL); Aharon WEISSBROD, Rehovot (IL); Danielle Ricca HONIGSTEIN, Rehovot (IL); Tali WEISS, Rehovot (IL); Lior GORODISKY, Rehovot (IL); Kobi SNITZ, Rehovot (IL); Aharon RAVIA, Rehovot (IL)

(73) Assignee: Yeda Research and Development Co. Ltd., Rehovot (IL)

(21) Appl. No.: 18/859,560

(22) PCT Filed: Apr. 24, 2023
(86) PCT No.: PCT/IL2023/050422

§ 371 Date: Oct. 24, 2024

Related U.S. Application Data
(60) Provisional application No. 63/334,328, filed on Apr. 25, 2022.

(51) Int. Cl. A61B 5/00; A61B 5/16

(52) U.S. Cl. CPC A61B 5/4848; A61B 5/168; A61B 5/7267

(57) ABSTRACT

A system for assessing an ADHD status of a subject comprising:
(a) receiving circuitry configured to receive a measurement signal including measurements of a plurality of nasal respirations from a sensor configured to sense a nasal respiration of a subject;
(b) optional determining circuitry configured to determine values of one or more nasal respiration parameter from said measurement signal;
(c) evaluation circuitry configured to evaluate said subject, based on said values of one or more respiration parameter to provide an indication of ADHD status of said subject.

### RELATED APPLICATION/S

This application claims the benefit of priority of U.S. Provisional Patent Application No. 63/334,328 filed on 25 Apr. 2022, the contents of which are incorporated herein by reference in their entirety.

### FIELD AND BACKGROUND OF THE INVENTION

The present invention, in some embodiments thereof, relates to screening, diagnosis and treatment monitoring for ADHD based on nasal respiration parameters.

Attention Deficit Hyperactivity Disorder (ADHD) is a neurodevelopmental disorder characterized by core symptoms of inattention, impulsivity, hyperactivity and diminished executive functions. ADHD is one of the most common neurodevelopmental disorders, affecting approximately 5% of children worldwide, with 60-80% of these patients having persistence of these symptoms into adulthood. The pathophysiology of ADHD is unclear and it appears to have a complex etiology. The diagnostic procedures of ADHD pursued by psychiatrists, neurologists, pediatricians and family practitioners are based largely, if not exclusively, on subjective assessments of perceived behavior. Owing to the subjective nature of the diagnostic criteria, ADHD may be misdiagnosed, thereby causing a delay in the appropriate treatment.

Additional Background Art includes:
U.S. application Ser. No. 17/380,348 now US patent publication 2023/0028914A1; PCT Application No. WO2021/209999;
Perl et al., Nature Human Behavior, 3, 501-512, 2019;
Zelano C et al., The Journal of Neuroscience, 36, 12448-12467, 2016;
Sonne et al., TEI 2016 Feb. 14-17, 2016, Eindhoven, The Netherlands; Sano et al., Neuroreport. 2013 Dec. 4; 24 (17): 935-940; and
Muñoz-Organero, M., Powell, L., Heller, B., Harpin, V., & Parker, J. (2018). Automatic Extraction and Detection of Characteristic Movement Patterns in Children with ADHD Based on a Convolutional Neural Network (CNN) and Acceleration Images. Sensors (Basel, Switzerland), 18(11). doi: doi(dot)org/10.3390/s18113924.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A system for assessing an ADHD status of a subject comprising:
receiving circuitry configured to receive a measurement signal including measurements of a plurality of nasal respirations from a sensor configured to sense a nasal respiration of a subject;
determining circuitry configured to determine values of one or more nasal respiration parameter from said measurement signal;
evaluation circuitry configured to evaluate said subject, based on said values of one or more respiration parameter to provide an indication of ADHD status of said subject.

Example 2. A system according to example 1, comprising at least one nasal flow sensor which provides said measurement signal.

Example 3. The system of example 1 or example 2, wherein said plurality of nasal respirations comprises at least two consecutive nasal respirations of the subject.

Example 4. The system of any one of examples 1-3, wherein said measurement signal includes at least 10% of all breaths over a time period of at least five minutes.

Example 5. The system of any one of examples 1-4, wherein said at least one respiration parameter comprises a volume-associated parameter.

Example 6. The system of any one of examples 1-5, wherein said at least one respiration parameter comprises a timing-associated parameter.

Example 7. The system of example 6, wherein said timing-associated parameter comprises at least one of a duration of a nasal inhalation, a duration of a nasal exhalation, a time between two nasal inhalations, a duty cycle and a time between two nasal exhalations.

Example 8. The system of any one of examples 1-7, wherein said at least one respiration parameter comprises at least one of a breathing rate, an inter-breath interval, an inhale volume, an exhale volume, a tidal volume, a minute ventilation and a duty cycle.

Example 9. The system of example 8, wherein said at least one parameter comprises inhalation and/or exhalation velocity and/or duration

Example 10. The system of any one of examples 1-9, wherein said evaluating comprises evaluating one or both of a coefficient of variation of said parameter over a predetermined time period and a peak value of said parameter over a predetermined time period.

Example 11. The system of any of examples 1-10, wherein said sensor is a pressure sensor.

Example 12. The system of any one of examples 1-11, wherein said evaluation circuitry is configured to monitor at least one parameter of a nasal respiratory waveform of the subject.

Example 13. The system of any of examples 1-12, wherein said evaluation circuitry includes a classifier trained to classify nasal breathing data into ADHD status.

Example 14. The system of any one of examples 1-13, wherein said ADHD status comprises an ADHD sub-classification or an ADHD severity score.

Example 15. The system of example 14, wherein said sub-classification of ADHD is selected from the group consisting of inattentive, hyperactive-impulsive, and combined inattentive hyperactive-impulsive.

Example 16. The system of any one of examples 1-15, wherein said evaluation circuitry is configured to compare said value of said at least one respiration parameter with a reference value, wherein a difference between said value of at least one respiration parameter of said subject and said reference value is indicative of a status of ADHD.

Example 17. The system of any of examples 1-16, comprising memory storing therein at least one reference value personalized for the subject.

Example 18. The system of example 17, wherein said at least one reference value includes at least two reference values, for different conditions of the subject.

Example 19. The system of any of examples 1-18, wherein said indication comprises a diagnosis of ADHD.

Example 20. The system of any of examples 1-19, wherein said indication comprises a change in status of ADHD.

Example 21. The system of any of examples 1-20, wherein said indication comprises a recommendation to take medication or apply a therapy.

Example 22. The system of any of examples 1-21, wherein said indication comprises an indication regarding an effectiveness therapy.

Example 23. The system of any of examples 1-22, wherein said determining circuitry or said evaluating circuitry is remote from said receiving circuitry.

Example 24. The system of any of examples 1-23, comprising a UI component configured to one or both of display an ADHD status to the subject and receive an input from the subject.

Example 25. The system of any of examples 1-24, comprising at least one additional sensor configured to sense environmental data and/or physiological data of the subject and wherein said evaluation circuitry is configured to take such data into account in said evaluating.

Example 26. The system of any of examples 1-25, comprising prediction circuitry configured to predict a future ADHD state based on medication data regarding medication taken by the subject.

Example 27. The system of any of examples 1-26, comprising timing circuitry configured to automatically cause said receiving circuitry and said determining circuitry and said evaluating circuitry to activate according to a schedule or in response to a trigger.

Example 28. A method of determining an Attention Deficit Hyperactivity Disorder (ADHD) status of a subject comprising:
measuring a plurality of nasal respirations of the subject, and
determining a value of at least one respiration parameter of said plurality of nasal respirations,
generating an indication of the ADHD status of the subject based on said value of said at least one respiration parameter.

Example 29. A method according to example 28, wherein said measuring comprises measuring at least two consecutive nasal respirations of the subject.

Example 30. A method according to example 28 or example 29, wherein said measuring comprises measuring at least 10% of the breaths of the subject over a period of at least ten minutes.

Example 31. A method according to any one of examples 28-30, wherein said measuring comprises measuring in a timed relationship to taking of ADHD-related medication by the subject.

Example 32. A method according to any one of examples 28-31, wherein said measuring comprises measuring while said subject is awake.

Example 33. A method according to any one of examples 28-32, wherein said measuring comprises measuring while said subject is asleep.

Example 34. A method according to any one of examples 28-33, wherein said measuring comprises measuring in two nostrils and comparing measurements between nostrils to identify ADHD status.

Example 35. A method according to any one of examples 28-32, wherein said measuring comprises automatically measuring at least two times in one day, at least 1 hour apart.

Example 36. A method according to any one of examples 28-35, wherein said generating comprises generating by applying a pre-trained classifier on said at least one respiration parameter.

Example 37. A method according to any one of examples 28-36, comprising generating an indication of ADHD status and/or change in ADHD status.

Example 38. A method according to any one of examples 28-36, comprising collecting baseline data for the subject and using said baseline data for said generation.

Example 39. A method according to example 38, wherein said baseline data comprises one or more of data for a group of comparable subjects and data from said subject.

Example 40. A method according to example 39, wherein said data comprises one or both of data for a healthy state, data for an ADHD state and data for a ADHD state which is treated.

Example 41. A method of determining whether a therapy or therapeutic agent is effective in managing ADHD in a subject comprising:
applying the therapy to the subject or administering the therapeutic agent to the subject; and
determining a status of ADHD in a time window shorter than 15 minutes, wherein the indication of ADHD is a responsiveness to the therapy or therapeutic agent, wherein said applying or administering is effected prior to or during the measuring.

Example 42. A method according to example 41, wherein said applying comprises indicating such applying or administering to a system used to measure the ADHD status.

Example 43. A method of monitoring the progression of ADHD in a subject comprising: determining, at a plurality of times, a status of ADHD in a time window shorter than 15 minutes, wherein the indication of ADHD is a progression of the ADHD between a first time of said plurality of times and a later time of said plurality of times.

Example 44. A method according to example 43, comprising predicting an expected increase in ADHD severity and a timing for preventive treatment thereof.

Example 45. A method of managing ADHD in a subject in need thereof comprising:
measuring a value of at least one timing and/or volume associated parameter of a plurality of nasal respirations of the subject over a time period,
communicating, during said time period, to the subject, when said value is indicative of ADHD, to alter nasal respirations such that said value of at least one timing and/or volume associated parameter is more similar to a control value which is not indicative of ADHD; and/or
communicating, during said time period, to the subject, when said value is not indicative of ADHD, to retain nasal respirations such that said value of at least one timing and/or volume associated parameter remains similar to a control value which is not indicative of ADHD.

Example 46. A system for assessing ADHD status of a subject comprising:
at least one sensor configured to sense nasal respirations of said subject;
a processor coupled with said at least one sensor, said processor being configured to analyze said nasal respirations;
a user interface configured to indicate an ADHD status or guidance based on an existing or predicted ADHD status to the subject.

Example 47. The system of example 46, wherein comprising a memory with subject-specific data which said processor is configured to retrieve for use for evaluating said subject using said sensed nasal respiration and wherein said ADHD status is evaluated based on said data.

Example 48. Use of a medication for treatment of ADHD with a dose and timing determined using an evaluation of patient ADHD status from nasal respiration.

Example 49. A computer readable media having thereon a classifier for ADHD based on nasal breathing parameters.

Example 50. A method of creating a classifier for ADHD status based on nasal breathing parameters, comprising: collecting nasal breathing parameters of a subject and training a machine learning system with said data using an indication of a known ADHD status of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave.

Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as assessing a subject, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E are graphs illustrating an increase in Peak Nasal Airflow in ADHD patients as compared to healthy subjects as measured using a pressure sensor, in accordance with some embodiments of the invention;

FIG. 2A is a graph illustrating exhibits the Nasal Cycle shifts between “ON” and “OFF” Ritalin, when measured using a system in accordance with some embodiments of the invention;

FIG. 2B is a graph illustrating change in nasal exhalation and inhalation amplitude shifts between “ON” and “OFF” Ritalin, when measured using a system in accordance with some embodiments of the invention;

FIG. 3 is a graph illustrating the correlation between the respiratory data and ADHD questionnaire score, when measured using a system in accordance with some embodiments of the invention;

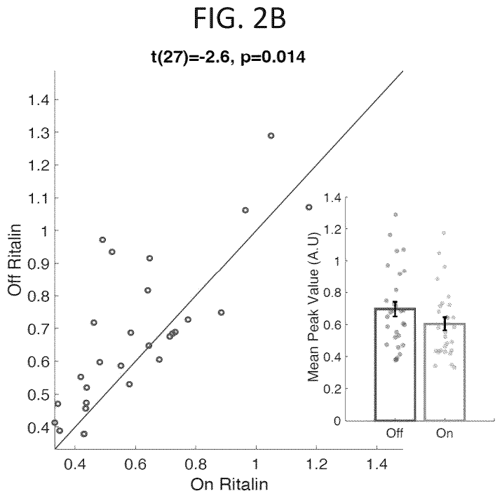

FIG. 4 is a graph illustrating identification of 5 among 6 ADHD subjects and 4 among 6 controls, using a neural network classifier based on nasal airflow parameters, in accordance with some embodiments of the invention;

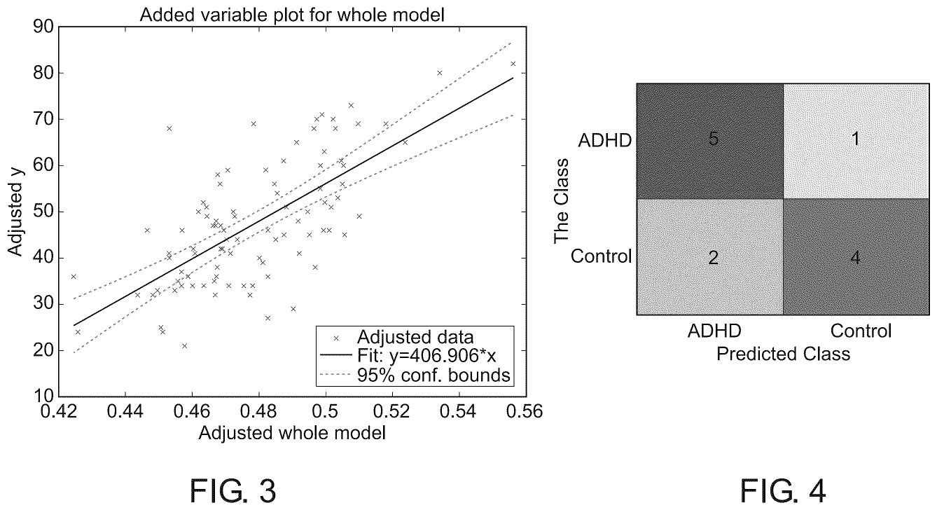

FIG. 5 is a graph illustrating that a 5 minute recording is sufficient to determine whether a subject is on or off Ritalin, when measured using a system in accordance with some embodiments of the invention;

FIGS. 6A-B are representative samples of nasal airflow recordings from a healthy subject, and processed in accordance with some embodiments of the invention;

FIG. 7 is a flow chart of a process for determining status of ADHD of a subject, according to some exemplary embodiments of the invention;

FIG. 8A is a flow chart of a process for determining responsiveness of a subject to a treatment for ADHD, according to some exemplary embodiments of the invention;

FIG. 8B is a flow chart of a process for ongoing subject monitoring, according to some exemplary embodiments of the invention;

FIG. 9A is a block diagram of a system for determining status of ADHD, according to some exemplary embodiments of the invention;

FIG. 9B shows an image of an exemplary nasal measuring system mounted on a subject and exemplary measurements and an optional mobile telephone used as a user interface, in accordance with some embodiments of the invention;

FIG. 10 is a flow chart of a process for monitoring progression of ADHD of a subject over time, according to some exemplary embodiments of the invention; and

FIG. 11 is a flow chart of a process for altering ADHD status of a subject by providing feedback to the subject regarding nasal respirations, according to some exemplary embodiments of the invention.

### DESCRIPTION OF SPECIFIC EMBODIMENTS OF THE INVENTION

The present invention, in some embodiments thereof, relates to diagnosis of ADHD and treatment monitoring for ADHD based on nasal respiration parameters. Such methods may be used for, for example, providing an objective markers of ADHD that might be used for diagnosis, prognosis and/or assessment of response to pharmacological interventions.

A broad aspect of some embodiments relates to determining the status of Attention Deficit Hyperactivity Disorder (ADHD) status of a subject based on nasal respiration measurements. In some embodiments of the invention, the status is an instantaneous status, for example, reflecting an hour, 20 minutes, 10 minutes, 5 minutes or smaller or intermediate time scales. Such a status may also be used to infer an overall problematic condition of the subject. It is noted that some subjects may exhibit an ADHD state only part of the time. In some subjects, an ADHD-like state may be desirable, for example, for certain activities. In some embodiments of the invention, the state is on a scale rather than binary and increase and/or decrease in the state may be noted.

The present inventors have shown that values and/or changes in values of nasal respiration parameters are indicative of ADHD status. Analysis of these parameters allow the user to obtain feedback (e.g. substantially instantaneous feedback) regarding ADHD status.

In some embodiments, the measuring is carried out using a portable/mobile device that is inserted inside or close to at least one nostril of the subject. The device may be retained at the site of measurement and/or on the subject's body without additional equipment or personnel, e.g. using a strap, a halter etc. In some embodiments of the invention, the device and/or other system components carried by the subject are mobile, for example, weighing less than 1 kg, 500 gr and smaller or intermediate weights. Optionally or additionally, some of the components are dual use, for example, a user-interface (UI) and/or some processing and/or communication functionality are provided by a mobile telephone or other mobile device used by the subject for other activities. The device itself may comprise circuitry which analyzes parameters of the nasal respiration signals and optionally further comprises circuitry that communicates the status of the ADHD based on values of these parameters. Alternatively, the device may be dedicated to measuring nasal respiration signals (e.g. nasal airflow) and may comprise circuitry such that it is connectable to an external processor/processors (e.g. on a mobile device such as a mobile telephone) which carry out the function of analysis and/or status communication. A potential benefit of using such a device (and optional mobile processor) to assess ADHD status of a subject is the relative simplicity of equipment and/or testing technique/s required e.g. as opposed to imaging methods e.g. structural and/or functional brain imaging e.g. neuroimaging and electrophysiology. Since measurement of nasal respiratory parameters was shown to be sufficient for providing an indication of ADHD status (as opposed to oral respiratory parameters), the subject is able to continue monitoring ADHD status during everyday activities, particularly ones in which physical movement is not required. In one embodiment, the subject is stationary during the measurement. For example, the subject may be in a classroom setting, an office setting or in the home.

In some embodiments of the invention, the device includes a user-interface allowing a subject to enter information (such as taking of drugs) and/or allowing the system to provided ADHD status and/or other guidance. Optionally, the UI is used to carry out various interactive methods as described herein, where the subject performs some activities (e.g., data entry) and the processor others (e.g., determining ADHD state). The device may store personalized attributes of the subject and/or include sensors to collect environmental and other data. As noted, some functions of the device may be carried out by associated devices of a system for ADHD subjects, for example, processing, UI and/or sensing may be provided by a cellular telephone. Optionally or additionally, processing may be provided by a remote server (e.g., a cloud server).

A potential benefit of using nasal respiration measurements to assess ADHD status of a subject is the relative speed in obtaining the assessment. As opposed to current diagnostic means which rely on questionnaires, computer-aided testing and interviews by professional psychologists, the present means of assessing ADHD can be carried out in a time-frame of minutes. The ability to determine ADHD status in a relatively short amount of time, and in a setting which does not require medical intervention, allows the subject to monitor the status of his/her ADHD during the day (e.g. in real-time) and determine for him/herself when and/or if medication is required and maintain his/her attention at the necessary level for his/her daily life. It will be appreciated that the ability to fine-tune and/or personalize treatment regimens of ADHD medication according to particular needs allows the use of medications which have a shorter half-life and/or potentially reducing unwanted side-effects, such as head-aches, appetite suppression etc. Optionally, the ADHD monitor system includes a prompting to advise the subject to modify drug intake and/or perform other activities. In some embodiments of the invention, the system includes personalized drug response data which may be used (e.g., by a processing function in the system), for example, to predict when a next dose may be needed and/or to allow comparing of a current measurement against a baseline. Such prediction may use, for example, general tables or patient-specific tables, which may link, ADHD status, optionally measurements that indicate ADHD status, medication dosage and timing of such dosage. Such tables may be calculated and/or may be tuned for a particular patient based, for example, on measurements of the effect of medication, for example determined using a system as described here.

In some embodiments, assessment of ADHD status of a subject using nasal respiration parameter/s is performed repetitively over time, for example, to assess subject progression over time. For example, to assess effectiveness of treatment e.g. over time, for example, as further described below. In this context, over time includes over a treatment session, for example detecting that efficacy of a drug is wearing off and/or over one or more days, weeks or months, to track progress of a subject.

In some embodiments, nasal respiration parameter/s monitoring is used during and/or with treatment and/or breathing protocol of a subject, e.g. to assess effectiveness of the treatment, as further described below.

In some embodiments, nasal respiration parameter/s are used to select treatment and/or a potentially therapeutic breathing protocol. For example, in some embodiments, a breathing protocol and/or treatment (e.g. medication given) is performed on a subject, and the nasal respiratory parameters are assessed. In some embodiments, personalized treatment plans are built using nasal respiratory assessment. In one example, a nasal monitoring system is used for training a suggest in applying certain, non-ADHD breathing patterns and/or to suggest to a subject to apply such patterns and/or track such pattern as they are being applied by a subject and provide feedback and/or guidance, e.g., as needed.

In some embodiments, nasal respiration parameter monitoring are performed repetitively, e.g. to provide a log of patient progress, optionally along with treatment data.

Additionally, or alternatively, using the methods described herein, the subject and/or the system may be able to identify triggers which set off ADHD in daily life.

In accordance with some embodiments of the invention, there is provided a method for processing physiological data, comprising: (a) collecting nasal respiration data from a subject; (b) using a pre-trained machine learning model to process the nasal respiration data to obtain a processed signal; and (c) using the processed signal to identify an ADHD status of the subject.

In accordance with some embodiments of the invention, there is provided a method building a ML model, such as a classifier, comprising; (a) collecting nasal respiration data from a plurality of subjects; (b) providing instant ADHD status information, for example, based on taking of medication or a questionnaire, which may be used for tagging the collected data; and (c) training machine learning model, such as a classifier using the tagged data. to identify an ADHD status of the subject. Optionally, additional data is used, such as patient status and physiological parameters/measurements other than nasal breathing. This data may be used for training and/or for normalizing the measured data. In some embodiments of the invention, an existing model is fine tuned for a particular subject.

In some embodiments of the invention, the data is cleaned before training, for example, by outlier removal.

In some embodiments of the invention, a learning system is used to collect data about a relationship between medication dosage, triggers and/or time and ADHD status. This may be personalized for a subject or for a group of subjects.

Also provided is a software system (and/or hardware system) which uses a classifier trained as above.

In some embodiments of the invention, the training uses one of the 25 parameters as described herein. Optionally, the input data is a variant of such parameters, for example, a parameter (e.g., extracted from nasal measurements) that has a high correlation with parameters as subjected herein, for example, above 50% or 75%.

In some embodiments of the invention, an ADHD-status system, for example, as described herein, is used for setting a medication dose for a subject. In general, such method may include collecting information about effect of medication dose on a subject over time and using this information to predict when a dose will have reduced effect and/or which dose can provide the desired ADHD relief at that time.

For example, a patient may be diagnosed with ADHD (e.g., using system 200 or using other methods, for example as known in the art).

Optionally, nasal measurements (or other data for methods of identifying instant ADHD state) are collected (e.g., using system 200) at least for an “on” state and for an “off” state of Ritalin effect. Optionally, the state of maximum and minimal effect are collected.

The subject is given a clinically therapeutic amount of drug, for example Ritalin.

Then, for a period of time, for example, several hours (e.g., until the Ritalin effect on ADHD is expected to weaken or wear off), for example continuously or periodically, for example, as described here. This creates a timeline of ADHD state, or at least ADHD parameters relative to a medication dosage (and optionally other information, such as triggers or environment).

Such indicators may be used to determine a binary ADHD state, and, optionally, intermediate ADHD states.

Evaluation of ADHD state may also be undertaken for the same time line using non-nasal methods, for example, a TOVA test or using a questionnaire.

The ADHD evaluation may be used to generate an overall timeline (or other relationship, such as a formula or parameters for such) linking an ADHD evaluation and time.

When the timeline indicates that ADHD state is about to become a disability state, more medication may be given and its effect tracked. Optionally, the medication is given before the ADHD state deteriorates too much. Optionally, the timing depends on the measured effect (e.g., measured using system 200) of the new dosage on the subject. During a titration period, various dosages and timings may be tried on a subject to learn various set points, so as to determine a best dosage. Optionally or additionally, measurements from one or more subjects are used for timing dosage for other subjects. Such measurements may also include tracking of side effects (e.g., to be reduced), possibly using a sensor 204 and/or prevent peak blood levels from being reached.

Thereafter, in ordinary use, a subject may be given prompts (e.g., by an app on his cellphone or from a remote location) to take more medication and at what dose. Optionally or additionally, the patient may wear a measuring system, such as system 200, to measure actual ADHD state, for later use and/or for suggesting medication timing.

In some embodiments of the invention, a classifier for ADHD-score is created by training. Optionally, data for different ADHD scores (e.g., using a TOVA test or a questionnaire) are used to tag subjects as having a certain score and then the classifier is trained on data for such subjects to yield the desired score.

In some embodiments of the invention, the methods described herein for tracking ADHD status are used over a period of 1-5 months or years, or longer. The improvement or worsening of ADHD score and/or response to medication may be used for prognosis, for example, based on prognosis of patients with similar etiologies and/or based on a monotonic (e.g., things will continue improving or worsening) or an asymptotic (e.g., if change is getting smaller, change will plateau and an asymptote will be reached) assumption

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Exemplary General Process for Determining Status of ADHD

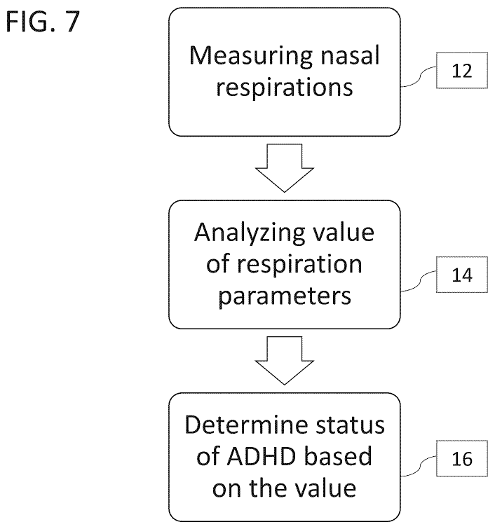

Reference is now made to FIG. 7, depicting a process for determining an ADHD status of a subject, (and optionally predicting a future ADHD status), according to some exemplary embodiments of the invention.

According to some exemplary embodiments, nasal respirations (e.g. nasal airflow) are recorded at block 12. In some embodiments, additional respiratory signals are recorded from the mouth.

In some embodiments, respiration is measured using one or more movement sensor and/or pressure sensor and/or one or more optical sensor.

For example, in some embodiments, a pressure sensor senses changes in pressure on the sensor associated with respiration. In some embodiments, pressure sensor is held in contact with and/or in position close to at least one nostril of the subject e.g. by a strap and/or adhesive.

Exemplary devices for measuring nasal respiration are known in the art and some are described in more detail herein below. However, other devices for measuring nasal respirations maybe used as well.

In some embodiments of the invention, the measuring is carried out whilst the subject is awake. Alternatively or optionally, the measuring is carried out whilst the subject is asleep.

In some embodiments, the measuring is carried out whilst the subject is stationary.

The subject may be of any age, including children (e.g. under the age of 18).

In one embodiment, a plurality of nasal respirations (e.g., of a same nostril) are measured. The nasal respiration may be measured at a single nostril or both nostrils. In one embodiment, the airflow in one nostril is measured independently of the airflow in the second nostril. In one embodiment at least two consecutive nasal respirations are recorded. In another embodiment at least 3, 4, 5, 6, 7, 8, 9, or 10 nasal respirations (optionally consecutive) are recorded. For example, measurement of 1-100 respirations, or 10-100 respirations, or 50-100 respirations, 50-500 respirations, or 5000-20,000 respirations, or 1-30 minutes, or 1-20 minutes, or 5-30 minutes, or 30 minutes-24 hours, or 2-8 hours, or about 6 hours, or about 24 hours are envisaged. A potential advantage of shorter times is a faster tracking of ADHD state. A potential advantage of longer times is longer term monitoring and/or reduced noise. It is noted that some nasal measurements may be skipped or not collected. For example, measurement may be on a sampling basis. Optionally or additionally, an accelerometer or other sensor (e.g., as will be described below) may be used to detect movement of the subject and such measurements ignored. Similarly, heart rate may be used as an indication to ignore some measurements or otherwise process them differently, for example, heart rate changes indicating an increase in activity.

It is a particular feature of some embodiments of the invention that as evaluation of ADHD status is rapid, data can be skipped in a more cavalier fashion and still result in rapid and/or quality evaluation. For example, fewer than 80%, 60%, 50%, 30%, 15% or 10% of breathing cycles may be measured and/or utilized in a time period (e.g., with at least 5, etc. respiration used).

In some embodiments, one or more nasal respiration parameter is determined continuously e.g. using continuous respiration measurements. In some embodiments, the subject is assessed using these respiration parameter/s continuously. Alternatively or additionally, in some embodiments, continuous respiration measurements are used to assess the subject periodically. As used herein, continuously includes collecting data and/or updating an ADHD status more often than once in 15 minutes, once in 10 minutes, once in 5 minutes, once a minute or intermediate update rates.

It is a particular feature of some embodiments of the invention, that ADHD status (and/or a change therein) can be determined within a time window of, for example, less than 15 minutes, less than 10 minutes, about or less than 5 minutes or shorter or intermediate times. This potentially allows rapid feedback to therapeutic processes and/or to obtain a finer grained understanding of a subject's ADHD journey and/or potentially predictive methods.

In some embodiments, a subject is assessed using measured nasal respiration parameters periodically. In some embodiments, respiration is measured (or continuous measurements are sampled) for short periods of time e.g. on a regular basis, for example, for 5-30 mins, 1-3 times a day.

It will be appreciated that the number of measurements and/or length of time during which the nasal respirations are recorded may be adapted according to the ADHD status that is being measured.

In one embodiment the measuring is effected in at least 10%, 20%, 30%, 40%, 50%, 60%, 70%, 80%, 90% or even 100% of all breaths over a time period of at least five minutes.

In one embodiment the measuring is effected in at least 10%, 20%, 30%, 40%, 50%, 60%, 70%, 80%, 90% or even 100% of all breaths over a time period of at least ten minutes.

In one embodiment the measuring is effected in at least 10%, 20%, 30%, 40%, 50%, 60%, 70%, 80%, 90% or even 100% of all breaths over a time period of at least twenty minutes.

In one embodiment the measuring is effected in at least 10%, 20%, 30%, 40%, 50%, 60%, 70%, 80%, 90% or even 100% of all breaths over a time period of at least thirty minutes.

In one embodiment the measuring is effected in at least 10%, 20%, 30%, 40%, 50%, 60%, 70%, 80%, 90% or even 100% of all breaths over a time period of at least one hour.

In one embodiment the measuring is effected in at least 10%, 20%, 30%, 40%, 50%, 60%, 70%, 80%, 90% or even 100% of all breaths over a time period of at least 2-10 hours.

In still further embodiments, the measuring is effected for at least 5, 10, 15, 20, 30, 45, 60 consecutive minutes or longer.

It will be appreciated that the measuring may be carried out at least twice a day, wherein each measurement event is effected in at least 10%, 20%, 30%, 40%, 50%, 60%, 70%, 80%, 90% or even 100% of all breaths over a time period of at least five minutes. The interval between the two measurement events is at least one hour, at least two hours, at least three hours, at least four hours, at least five hours, at least six hours or longer or intermediate rates.

According to some exemplary embodiments, a value of at least one respiration parameter of nasal respirations are determined (e.g., recorded) at block 14.

In some embodiments, a respiration parameter characterizes one or both of inhalation and exhalation, for example, for a time period which is of longer duration than a single respiration.

In some embodiments, each respiration includes features including for example, the respiration trace itself, volume, duration, and peak airflow for each portion of the respiration, where portions include, for example, inhalation and exhalation.

In some embodiments, respiration parameters include average magnitude of respiration feature/s and/or, peak respiration feature/s and/or variability of respiration feature/s over time.

According to a particular embodiment, the value of the respiration feature is a peak value of the feature or a mean peak value of the feature over the course of a predetermined number of respirations or over the course of a predetermined time (e.g. five minutes).

According to a particular embodiment, the value of the respiration feature is a coefficient of variation of the feature over the course of a predetermined number of respirations or over the course of a predetermined time (e.g. five minutes).

Exemplary respiration parameters include timing related parameters and volume related parameters. These may include, for a measured time period:
variability of the respiration measurement;
average and variability, for inhalation and/or exhalation, for one or more of; timing, peak airflow speed and volume.

According to a particular embodiment, at least one, at least two, at least three, at least four, at least five, at least six or all of the following respiration parameters are analyzed:
Breathing rate (e.g. number of inspirations per minute);
Inter-breath interval (e.g. average time between inhale onsets);
Inhale volume e.g. sum of airflow between inhale onset and offset (e.g. calculated as integral of the signal);
Exhale volume e.g. sum of airflow between exhale onset and offset (e.g. calculated as integral of the signal;
Tidal volume (e.g. average volume of air displaced per breath, which can be calculated as average inhale volume+average exhale volume);
Minute ventilation (e.g. volume of air displaced each minute, which may be calculated from breathing rate x average tidal volume); and/or
Duty cycle (e.g. proportion of breath that is inhaled-Standard deviation of inhale duration/average inhale duration.

Additional respiration parameters that may be analyzed include for example, as follows. It is noted that Volume and Duration appear to be useful parameters with significant predictive value. Pauses may also be analyzed, for example, to detect prevalence and/or length of such pauses.

The following are 25 parameters including matlab code for calculation thereof. These parameters are used in example 2, below.

$$\begin{matrix}1. & \\ Inhale\_Volume = mean ( [ only\_inhales . Volume ] ) ; & \end{matrix}$$

$$\begin{matrix}2. & \\ Exhale\_Volume = mean ( [ only\_exhales . Volume ] ) ; & \end{matrix}$$

$$\begin{matrix}3. & \\ Inhale\_Duration = mean ( [ only\_inhales . Duration ] ) ; & \end{matrix}$$

$$\begin{matrix}4. & \\ Exhale\_Duration = mean ( [ only\_exhales . Duration ] ) ; & \end{matrix}$$

$$\begin{matrix}5. & \\ Inhale\_value = mean ( [ only\_inhales . PeakValue ] ) ; & \end{matrix}$$

$$\begin{matrix}6. & \\ Exhale\_value = mean ( [ only\_exhales . PeakValue ] ) ; & \end{matrix}$$

$$\begin{matrix}7. & \\ Inter\_breath \_interval = mean ( diff ( [ only\_inhales . StartTime ] ) ) ; & \end{matrix}$$

$$\begin{matrix}8. & \\ Rate = 1. / [ Inter\_breath \_interval ] ; & \end{matrix}$$

$$\begin{matrix}9. & \\ Tidal\_volume = [ Inhale\_Volume ] + [ Exhale\_Volume ] ; & \end{matrix}$$

$$\begin{matrix}10. & \\ Minute\_Ventilation = [ Rate ] . * [ Tidal\_volume ] ; & \end{matrix}$$

$$\begin{matrix}11. & \\ Duty\_Cycle \_inhale = mean ( [ only\_inhales . Duration ] . / [ Inter\_breath \_interval ] ) ; & \end{matrix}$$

$$\begin{matrix}12. & \\ Duty\_Cycle \_exhale = mean ( [ only\_exhales . Duration ] . / [ Inter\_breath \_interval ] ) & \end{matrix}$$

$$\begin{matrix}13. & \\ COV\_InhaleDutyCycle = std ( [ only\_inhales . Duration ] ) . / mean ( [ only\_inhales . Duration ] ) ; & \end{matrix}$$

$$\begin{matrix}14. & \\ COV\_ExhaleDutyCycle = std ( [ only\_exhales . Duration ] ) . / mean ( [ only\_exhales . Duration ] ) ; & \end{matrix}$$

$$\begin{matrix}15. & \\ COV\_BreathingRate = std ( diff ( [ only\_inhales . StartTime ] ) ) . / [ Inter\_breath \_interval ] ; & \end{matrix}$$

$$\begin{matrix}16. & \\ COV\_InhaleVolume = std ( [ only\_inhales . Volume ] ) . / [ Inhale\_Volume ] ; & \end{matrix}$$

$$\begin{matrix}17. & \\ COV\_ExhaleVolume = std ( [ only\_exhales . Volume ] ) . / [ Exhale\_Volume ] ; & \end{matrix}$$

$$\begin{matrix}18. & \\ Inhale\_Pause \_Duration = mean ( inhale\_pause ) ; & \end{matrix}$$

$$\begin{matrix}19. & \\ Exhale\_Pause \_Duration = mean ( exhale\_pause ) ; & \end{matrix}$$

$$\begin{matrix}20. & \\ COV\_InhalePauseDutyCycle = std ( inhale\_pause ) . / mean ( inhale\_pause ) ; & \end{matrix}$$

$$\begin{matrix}21. & \\ COV\_ExhalePauseDutyCycle = std ( exhale\_pause ) . / mean ( exhale\_pause ) ; & \end{matrix}$$

$$\begin{matrix}22. & \\ Duty\_Cycle \_InhalePause = mean ( inhale\_pause . / [ Inter\_breath \_interval ] ) ; & \end{matrix}$$

$$\begin{matrix}23. & \\ Duty\_Cycle \_ExhalePause = mean ( exhale\_pause . / [ Inter\_breath \_interval ] ) ; & \end{matrix}$$

$$\begin{matrix}24. & \\ PercentBreathsWithExhalePause = length ( exhale\_pause ) * 100. / ( size ( peaks , 1 ) - size ( only\_inhales , 1 ) ) ; & \end{matrix}$$

$$\begin{matrix}25. & \\ PercentBreathsWithInhalePause = length ( inhale\_pause ) * 100. / ( size ( only\_inhales , 1 ) ; & \end{matrix}$$

According to some exemplary embodiments, the status of the ADHD is determined at block 16. The status is determined based on the valued obtained at block 14. It is noted that the status may include an indication of the status, but need not have a reliability of over 90%. In many uses, a reliability of, for example, 70%, 80% or higher or intermediate reliabilities (e.g., sensitivity) may be useful. For example, if the subject is being screened, a relative low false negative rate, even with a relatively high false positive rate may be good. In another example, if a subject is known to have ADHD, detecting changes in ADHD severity may be sufficient, as the absolute diagnosis is known.

A status of ADHD may refer, for example, to ADHD severity or a sub-classification of ADHD. The severity status of ADHD may correlate with known scales of severity—for example according to Conners rating scale, Swanson, Nolan and Pelham [SNAP]-IV) scale, ADHD-RS-V, Clinical Global Impression of Improvement or the ADHD Investigator Symptom Rating Scale.

Exemplary sub-classifications of ADHD include inattentive, hyperactive-impulsive, and combined inattentive hyperactive-impulsive. It is noted that during monitoring (as opposed to diagnosis), also small changes in an ADHD score are meaningful, so the ADHD status may be provided, in such cases, as a score with more than 4, 6, 10, 20 or intermediate numbers of values.

The ADHD status may be obtained by comparing the at least one respiration parameter value with a reference value, wherein a difference between the two is indicative of a status of ADHD. Optionally, the reference value is a personalized value for the subject. Alternatively, the reference value is a group reference value—e.g., for people of similar age and/or health status.

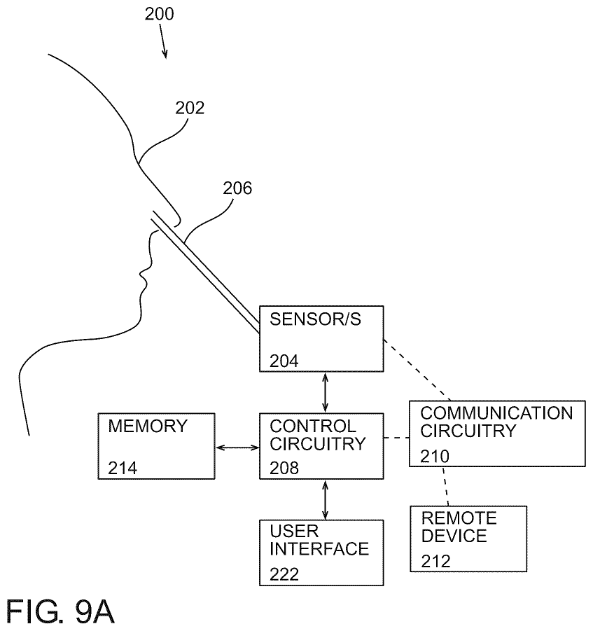

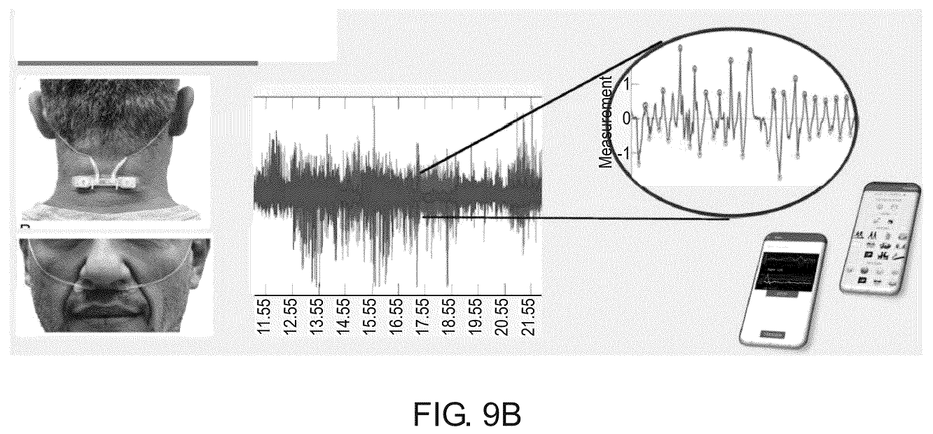

The comparison between the values can be made utilizing techniques such as reference limits, discrimination limits, or risk defining thresholds to define cutoff points and abnormal values. Comparison activities and/or data used (e.g., as reference) for such activities may be stored in an ADHD system, for example, as described herein with reference to FIG. 9A.

In some embodiments, one or more threshold is selected and/or adjusted and/or determined depending on a desired status e.g. of a patient and/or patient group. For example, one or more threshold is selected and/or adjusted and/or determined based on desired specificity and/or sensitivity. In some embodiments, a one or more threshold is selected and/or adjusted and/or determined based on relative importance (e.g. in the assessment) of specificity and/or sensitivity.

In one embodiment, the reference value is the value of a normal control value which is derived from a subject (or group of subjects) not suffering from ADHD. According to a particular embodiment, an average value derived from a group of subjects known to not be suffering from ADHD is used as the control value. Such normal control values and cutoff points may vary based on whether a value is used from a single respiratory parameter or in a formula combined with values from other respiratory parameters into an index. Alternatively, the normal control level can be a database of respiratory parameter patterns from previously tested subjects.

When the value of the respiratory parameter of the tested subject is sufficiently similar (e.g. statistically significantly similar) to a reference value derived from a non-ADHD subject (or group of subjects), it is indicative that the subject does not have ADHD. When the value of the respiratory parameter of the tested subject is sufficiently dissimilar (e.g. statistically significantly dissimilar) to a reference value derived from a non-ADHD subject (or group of subjects), it is indicative that the subject has ADHD.

In one embodiment, the reference value is the value of a control value which is derived from a subject (or group of subjects) suffering from ADHD. When the value of the respiratory parameter of the tested subject is sufficiently similar (e.g. statistically significantly similar) to a reference value derived from an ADHD subject (or group of subjects), it is indicative of ADHD in the subject. When the value of the respiratory parameter of the tested subject is sufficiently dissimilar (e.g. statistically significantly dissimilar) to a reference value derived from an ADHD subject (or group of subjects), it is indicative that the subject does not have ADHD.

Three particular ADHD measurement cases will be noted.

In a first use, a subject who may be suspected of having ADHD wears an ADHD system (e.g., such as system 200, below), for a period of time, possibly in a clinical setting and optionally while performing a task. Such task and measurement may be managed by a computer. Optionally, measurements are taken with and without medication, so as to determine not only ADHD status, but also which/if certain medication can assist in improving ADHD status. Alternatively or optionally, such use may be for a subject where it is suspected that certain foods or drugs or activities cause ADHD-like symptoms. The trigger is applied and ADHD status is monitored. In a clinical setting, the system need not be mobile and may be, for example, include a nasal sensor connected by a cable (directly or indirectly) to an electronics box, such as a desktop or laptop computer.

In a second use, screening, no prior suspicion regarding the subject status is used. Instead, the system is applied to the subject for a period of time sufficient to provide an indication of ADHD status. This can be repeated for multiple subjects one after the other. For example, this may be applied in a school or classroom setting. Again, the ADHD system used may be less mobile.

For screening and/or diagnosis, feedback re ADHD status may be provided to a user other than the subject.

In a third case, the ADHD system is used for monitoring and is worn by the patient for several hours or possibly a whole day (or more), while carrying out regular activities. The user may be prompted by the system to perform various activities (optionally including answering questions and/or performing cognitive or physical tasks) and/or the system may provide feedback on ADHD status to the subject and/or provide guidance.

In a variant of diagnosis, a subject may wear an ADHD system for several hours to track ADHD status over the day or during a long activity. Such ADHD system may be mobile, for example, as for monitoring.

Other uses are described herein as well.

### Exemplary System for Determining ADHD Status

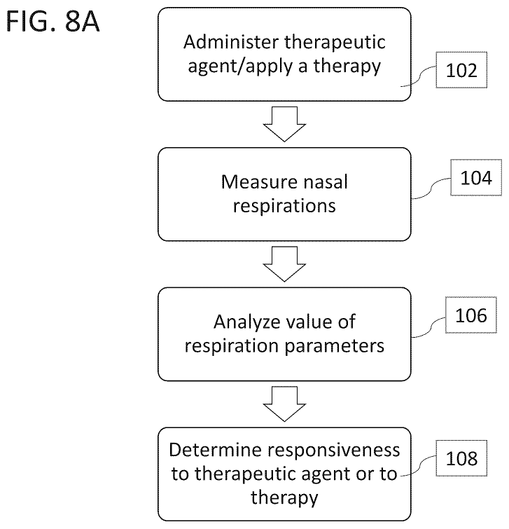

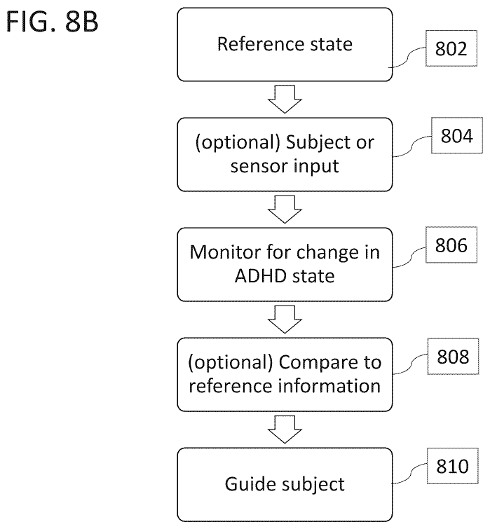

According to some exemplary embodiments, an ADHD status system, for example system 200 shown in FIG. 9A is used to determine an ADHD status of a subject and/or for other methods, for example, as described below with reference to FIGS. 8A, 8B, 10 and 11. The system may be programmed to control a UI and/or measurement, evaluation and/or communication circuitry as described in the methods. In some embodiments, nasal respiration is monitored by monitoring nasal airflow of at least one nostril of a subject using a device comprising one or more sensor 204 (e.g. a pressure or flow sensor).

In some embodiments, system 200 includes one or more additional sensor, e.g. for physiological measurement of subject. For example, one or more of a blood oxygenation sensor (e.g. located on a subject's finger), a temperature sensor, a cardiac cycle sensor. In some embodiments, an optical sensor detects and/or measures respiration (e.g., chest movement or nostril movement) and/or other subject parameters (e.g. other movements of the subject). Optionally, a smart watch or fitness sensor-type subsystem is used to collect physiological information. In some embodiments of the invention, sensor 204 includes an acceleration and/or other movement sensor to detect activity of the user (and optionally ignore measurements during movement). Alternatively, or optionally, sensor 204 includes an environmental sensor to detect environmental information such as audio (e.g., noise level), temperature, humidity and/or light. In some embodiments of the invention, sensors on a cellular telephone or other worn or hand-held electronic device is used to collect environmental and/or physiological information, for example, using sensing means and/or processing methods known in the art (e.g., microphone for sound, accelerometers for movement). It is noted that a typical cellular telephone has many sensors which may be repurposed, for example using methods know in the art, for collecting physiological and/or environmental data. Optionally, data collected by such sensors is used to calibrate the ADHD status, for example, for subjects where environmental factors (e.g., noise) increases ADHD-type behavior. In some embodiments of the invention, a user can access their data from an app on their mobile phone and/or download it form a cloud location or other remote server.

According to some exemplary embodiments, a nasal respiration monitoring system receives the signals recorded at the measurements sites, and generates an ADHD health indication, optionally in a form of index measurements. In some embodiments, the system presents the ADHD indication to a user.

According to some exemplary embodiments, the system comprises at least one circuitry, for example control circuitry 208, which processes the received signals. In some embodiments, the signal processing includes at least one of removing artifacts from the received signals and filtering of the received signals. In some embodiments, the received signals are processed using one or more algorithms formulas, and/or look-up tables (or other data) stored in a memory of the system, for example memory 214. Alternatively or additionally, the control circuitry processes the received signals using one or more algorithms, formulas and/or look-up tables and/or other data stored in a remote device 212. As can be seen, data processing may include multiple steps, including—signal processing to clear the signals, nasal parameter extraction from the optionally cleaned signals ADHD status determining from the extracted nasal parameter values and/or prediction of ADHD status and/or treatment suggestion, e.g., based on ADHD status. Additional processing in system 200 may include processing of non-nasal sensing, user interface management and process management. Each processing may be performed by separate processor in some embodiments. In others, two or more of these processing types are provided by a same circuitry (e.g., a processor). In an exemplary system the following processing loci may exist and one or more of them may be used: circuitry coupled to the sensors, a processor mounted on the subject's body, a mobile telephone, a cloud server. In some embodiments of the invention, the raw data or filtered data is used to directly indicate ADHD status, for example, by using a classifier trained on raw sensor data, rather than on extracted nasal flow parameters. Optionally, such a system is trained in two steps. First, a first classifier is generated using extracted nasal parameters. Once such first classifier is trained and/or validated, a new classifier can be trained on the raw data and receiving scoring of ADHD status using the trained classifier. Alternatively, a classifier may be trained on the raw data and using patient status indications. A potential advantage of the two step method is that more data can be made available as the first classifier can provided many data points for a single subject, as, as noted herein, ADHD classification can be rapid.

According to some exemplary embodiments, a control circuitry of the system 200 analyzes the processed signals. In some embodiments, the analysis comprises at least one of calculating power and/or phase relationships between processed signals received from sensor 204, optionally using one or more signal features. Optionally, the control circuitry applies at least classifier or other model or parameter set (e.g., generated by a machine learning algorithm and/or a neural network classifier) on the one or more signal features to determine an ADHD status, for example an ADHD diagnosis and/or determine responsiveness of a subject to a medication/therapy. In some embodiments, the control circuitry analyses the processed signals using one or more algorithms stored in the memory of the system, for example memory 214. Alternatively or additionally, the control circuitry analyses the processed signals using one or more algorithms, formulas and/or look-up tables stored in a remote device 212. Optionally, the control circuitry analyses the processed signal taking into account the body and/or head posture of the subject.

According to some exemplary embodiments, the control circuitry optionally generates a confidence index. In some embodiments, the confidence index is calculated based on the analyzed signals and/or based on one or more indications stored in the memory of the system. In some embodiments, values of the confidence index indicate a degree of confidence of the calculated values of the ADHD status.

Additionally, in some embodiments, the ADHD status is generated using one or more subject-related indication stored in a memory of the system and/or in the remote device. In some embodiments, the subject-related indication comprise indication regarding at least one of, clinical state of the subject, subject age, subject BMI, medical history of the subject and/or drug regime of the subject.

In some embodiments of the invention, the subject-related indication comprises base line information about the patient, for example, a calibration cure or thresholds.

According to some exemplary embodiments, at least one or all of the ADHD status indications are communicated to a user of the system, for example to the subject himself and/or to a professional (and/or recorded in an electronic medical record or other logging system). In some embodiments, the indications are delivered using a user interface, for example user interface 222 or using a display operationally connected to the user interface 222. An example user interface includes one or more buttons and one or more indicator lights and/or a speaker. In another example. A UI includes a display, for example, a touch-sensitive display and/or speakers and/or a microphone of a cellular telephone. UI 222 generally also includes circuitry to present and receiving information and actions.

In some embodiments, user interface 222 provides feedback to a user regarding sensor measurements. In some embodiments, user interface 222 prompts the user as to how to adapt nasal respiration or perform other activities, for example, therapeutic activities and/or diagnostic activities.

According to some exemplary embodiments, the system 202 comprises at least one communication circuitry 210 configured to transmit and/or to receive signals from at least one remote device, for example a remote device located at a distance larger than 1 meter from the system 202, a remote device located outside a room where the system 202 is located, a remote server, a cloud storage device, a remote database. In some embodiments, the at least one communication circuitry 210 is configured to transmit and/or receive wireless signals from the remote device 226, for example Bluetooth signals, Wi-Fi signals, and/or cellular signals.

According to some exemplary embodiments, the control circuitry is configured to process and/or to generate the information flow indication, using one or more algorithms stored in the remote device. In some embodiments, the control circuitry transmits the signals received from the sensors or indications thereof to the remote device, and received processed signals or the information flow indication from the remote device. Optionally the processing and/or the generation of the information flow indication is performed in the remote device 212 using one or more algorithms stored in the remote device 212.

FIG. 9B shows an image of an exemplary nasal measuring system mounted on a subject and exemplary measurements and an optional mobile telephone used as a user interface, in accordance with some embodiments of the invention. As can be seen, the nasal sensors may be on a strap that goes from the back of a subjects head to under the nostrils, where the sensors 204 may be located. Processing electronics may be at the nape of the neck and/or attached by a cable to such location.

### Exemplary Classifier

For example, in some embodiments, a logistic regression classifier is constructed based on all or subset of respiration parameters described in this document, including nasal respiration parameters such as breathing rate, an inter-breath interval, an inhale volume, an exhale volume, a tidal volume, a minute ventilation and a duty cycle. Where, in some embodiments, the classifier is constructed by choosing a cutoff value and classifying inputs with probability greater than the cutoff as one class, below the cutoff as the other. In some embodiments, the classifier detects a level of ADHD (e.g. VS/UWS vs. MCS) and/or predicts responsiveness to drug therapy (e.g. Ritalin) or neurofeedback therapy.

For example, in some embodiments, a classifier is constructed using an alternative machine learning technique. For example, one or more of Perceptron, Naive Bayes, Decision Tree, K-Nearest Neighbor, Artificial Neural Networks/Deep Learning, and Support Vector Machine.

In a particular example, the following MatLab code (2021b) is used for generating a classifier for ADHD status from nasal respiration data. Such classifier was used in Example 2 below.

```
% Code Overview and Comments
% ---------------------------
% 1. Load data files and prepare the dataset for analysis.
% 2. Loop through each subject, load their respective data files, and compute the mean
values for various measures.
% 3. Split the data into training and testing sets for ADHD and control groups.
% 4. Train a neural network classifier on the training data and evaluate its performance
on the testing data using accuracy and confusion matrix.
% 5. Perform cross-validation to estimate the classifier's validation accuracy.
%
% Note: The variable names used in this code are generally self-explanatory.
clear
% Load dataset
load(‘AllSubjData_175.mat’)
% Choose subjects
subject_to_use = importdata(‘./data/SubjectsToUse.xlsx’); % Subjects names
subject_to_use = subject_to_use.all_subj;
% Remove subjects not in the analysis
subjectsNamesToRemoveFromNasalCycleAnalysis = setdiff({AllSubjData.Name},
subject_to_use);
subjectsIndicesToRemoveFromNasalCycleAnalysis = cellfun(@(str) any(strcmp(str,
subjectsNamesToRemoveFromNasalCycleAnalysis)), {AllSubjData.Name});
AllSubjData = AllSubjData(~subjectsIndicesToRemoveFromNasalCycleAnalysis);
% Load quality assessment data
load(“./data/QA2_97.mat”); % A file with the ASRS grades
zscored = true;
block_length = 5;
WSA = ‘wake’;
mean_values_z = [ ];
number_of_permutations = 200
% Preprocess data
for sbj = 1:size(QA2, 2)
  SubjectName = AllSubjData(sbj).Name;
  SubjectGroup = AllSubjData(sbj).Group;
  if zscored
    file = dir([‘./data/’ SubjectName ‘/*zelano_’ WSA ‘_no_overlap_normalized.mat’]);
  else
    file = dir([‘./data/’ SubjectName ‘/*zelano_’ WSA ‘_no_overlap.mat’]);
  end
  load([file.folder ‘/’ file.name]);
  if size(mat) ~= size(measureResults)
    fprintf(‘check %s\n’, SubjectName)
  end
  [mat.Nostril_Corr_RValue] = measureResults.Nostril_Corr_RValue;
  [mat.Nostril_Corr_PValue] = measureResults.Nostril_Corr_PValue;
  [mat.MeanAmplitudeLI] = measureResults.MeanAmplitudeLI;
  [mat.MeanLateralityIndex] = measureResults.MeanLateralityIndex;
  [mat.stdAmplitudeLI] = measureResults.stdAmplitudeLI;
  [mat.stdLateralityIndex] = measureResults.stdLateralityIndex;
  names = fieldnames(mat);
  names = regexprep(names, ‘_’, ‘ ’);
  x = struct2table(mat);
  Data_per_subj = table2array(x);
  m_firstGroupValues = mean(Data_per_subj, ‘omitnan’);
  max_expected = m_firstGroupValues + 2.5 * std(Data_per_subj, ‘omitnan’);
  min_expected = m_firstGroupValues − 2.5 * std(Data_per_subj, ‘omitnan’);
  Data_per_subj(Data_per_subj > max_expected | Data_per_subj < min_expected) =
NaN;
  mean_per_subj = mean(Data_per_subj(:, :), ‘omitnan’);
  mean_values_z = [mean_values_z; mean_per_subj];
  clearvars mean_per_subj sbj
end
% Group data based on ADHD symptoms
A = {QA2.ASRS};
A(cellfun(‘isempty’, A)) = {NaN};
TA = cell2mat(A);
prc_to_train = 0.95;
ADHD_TH = 51
% Train and evaluate the neural network model
for perm = 1:number_of_permutations
      firstGroupValues = mean_values_z(TA > ADHD_TH, [1:25]);
      secondGroupValues = mean_values_z(TA < ADHD_TH, [1:25]);
      % Split data into training and testing sets
      sbj_to_train = randperm(size(firstGroupValues, 1), round(prc_to_train *
size(firstGroupValues, 1)));
      balanced_control = randperm(size(secondGroupValues, 1), size(sbj_to_train, 2));
      sbj_to_test = setdiff(1:size(firstGroupValues, 1), sbj_to_train);
      control_to_test = setdiff(1:size(secondGroupValues, 1), balanced_control);
      balanced_control_to_test = randperm(size(control_to_test, 2), size(sbj_to_test,
2));
      % Create training and testing matrices
      training_mat = [firstGroupValues(sbj_to_train, :);
secondGroupValues(balanced_control, :)];
      labels_training = [repmat({‘ADHD’}, size(sbj_to_train, 2), 1); repmat({‘control’},
size(sbj_to_train, 2), 1)];
      testing_mat = [firstGroupValues(sbj_to_test, :);
secondGroupValues(balanced_control_to_test, :)];
      labels_testing = [repmat({‘ADHD’}, size(sbj_to_test, 2), 1); repmat({‘control’},
size(balanced_control_to_test, 2), 1)];
      % Train neural network
      [mdl, validationAccuracyReal(perm)] = NeuralNetwork(training_mat,
labels_training);
      % Evaluate classifier
      labels_hat = mdl.predictFcn(testing_mat);
      C = confusionmat(labels_testing, labels_hat);
      C_{perm} = C;
      acc(perm) = sum(diag(C)) ./ sum(C(:));
end
% Define neural network training function
function [trainedClassifier, validationAccuracy, validationPredictions] =
mediumNeuralNetwork(trainingData, responseData)
ClassNames = unique(responseData);
classificationNeuralNetwork = fitcnet(...
predictors, ...
response, ...
‘LayerSizes’, 300, ...
‘Activations’, ‘relu’, ...
‘Lambda’, 0, ...
‘IterationLimit’, 1000, ...
‘Standardize’, true, ...
‘ClassNames’, ClassNames);
predictorExtractionFcn = @(x) array2table(x);
neuralNetworkPredictFcn = @(x) predict(classificationNeuralNetwork, x);
trainedClassifier.predictFcn = @(x)
neuralNetworkPredictFcn(predictorExtractionFcn(x));
trainedClassifier.ClassificationNeuralNetwork = classificationNeuralNetwork;
trainedClassifier.About = ‘This struct is a trained model exported from Classification
Learner R2022a.’;
trainedClassifier.HowToPredict = sprintf(‘To make predictions on a new predictor
column matrix, X, use: \n yfit = c.predictFcn(X) \nreplacing “c” with the name of the
variable that is this struct, e.g. “trainedModel”. \n \nX must contain exactly 36 columns
because this model was trained using 36 predictors. \nX must contain only predictor
columns in exactly the same order and format as your training \ndata. Do not include
the response column or any columns you did not import into the app. \n \nFor more
information, see <a href=“matlab:helpview(fullfile(docroot, “stats”, “stats.map”),
“appclassification_exportmodeltoworkspace”)”>How to predict using an exported
model</a>.’);
predictors = trainingData;
response = responseData;
% Perform cross-validation
partitionedModel = crossval(trainedClassifier.ClassificationNeuralNetwork, ‘KFold’, 5);
validationAccuracy = 1 − kfoldLoss(partitionedModel, ‘LossFun’, ‘ClassifError’);
end
```

It should be noted that data can be stored in blocks, for example, 5 minute blocks of data and then prediction and/or training can use as many blocks as desired. As shown below, the classifier shows good results already with 5 minutes of breathing data. The loaded user data may include the 25 (extracted) nasal respiration parameters listed herein.

In brief a neural network classifier is optionally used, which may be trained, for example, with training data included 95% (n−1) of the ADHD and a balanced control group, and the testing consist 1 control and 1 ADHD. As preprocessing the raw data is optionally z-scored, and then divided into 5 minutes intervals. Outliers/intervals are optionally excluded. In some embodiments of the invention, the respiratory parameters per block are calculated and then averaged together.

In some embodiments, input/s to a classifier includes respiration parameter/s (e.g. at least one, two three, four, five, six or all of the following parameters: breathing rate, an inter-breath interval, an inhale volume, an exhale volume, a tidal volume, a minute ventilation and a duty cycle.

Optionally, in some embodiments, inputs to the classifier include the subject's state of health (or activity, such as walking) with respect to expected effect on the subject's physiological breathing apparatus. For example, subjects having respiration related conditions e.g. asthma, emphysema, pneumonia and/or conditions likely to affect respiration e.g. heart disease, in some embodiments, are assessed using different respiration parameter/s and/or using a portion of classifier which has be generated using respiration parameter data for this type of subject.

For example, in some embodiments, (e.g. where respiration volume is likely to be affected by a medical condition of the subject and/or an age of the subject) volume respiration parameters are normalized before use in assessment of the subject.

In some embodiments, output/s of a classifier include a probability that the subject is functioning at a particular level of ADHD. In some embodiments, output/s of classifier include an indication regarding the subject responding to therapy, for example, based on responsiveness of other subjects with similar nasal parameters. Optionally or additionally, the classifier outputs an ADHD score, as a number on a scale.

In some embodiments, the classifier determines a probability that a subject is in a group of a particular ADHD status using one or more respiration parameter including in some embodiments, only nasal respiratory parameters and, in some embodiments, both nasal respiratory and oral respiratory parameters. Where, in some embodiments, different parameters are weighted by the classifier.

Exemplary Process for Determining Responsiveness of a Subject Suffering from ADHD to a Therapy

Reference is now made to FIG. 8A, depicting a process for determining responsiveness of an ADHD subject to a treatment, according to some exemplary embodiments of the invention. This process is particularly useful for identifying personalized treatments for a subject.

According to some exemplary embodiments, a therapeutic agent or therapy is provided to a subject at block 102. The therapeutic agent may be an agent known to be generally useful in managing ADHD (e.g. FDA approved drug). Examples of such agents include Methylphenidate or derivatives thereof, isdexamfetamine, dexamfetamine, atomoxetine, guanfacine and amphetamine. Drugs which comprise Methylphenidate include Ritalin™ and Concerta™. Drugs which include amphetamine include Adderall™ and Vyvanse™. It will be appreciated that the process described herein may be useful for determining therapeutic effect of candidate agents, whose activity is yet to be determined.

The present application also includes new dosage protocols for existing drugs, for example, timing personalized according to ongoing ADHD measurements. This may result in on-demand taking of a dosage and/or in planning new dose regimens for a subject, for example, based on a typical reaction to a drug and/or allowed maximum blood levels, a new regimen may be planned per subject which provided increased anti-ADHD activity when needed. In some embodiments of the invention, the regimen is designed around daily activities, for example, tapering off a stimulant effect (e.g., based on pharmacodynamic considerations) when in a resting period and increasing anti-ADHD activity (e.g., based on a prediction based on current or previous reactivity, for times when more concentration is needed (e.g., math classes).

Exemplary therapies that may be provided to the subject include brain training, neurofeedback, breathing protocols etc.

Nasal respirations are measured at block 104. Measurements may occur simultaneously with the start of treatment or after a predetermined amount of time such that a therapeutic agent brings about the required effect. Alternatively, measurements may occur throughout a treatment protocol (e.g. during a breathing protocol). Alternatively or optionally, measurement starts before treatment so as to collect baseline state information.

Values of nasal respiratory parameters are analyzed at block 106. Exemplary parameters of nasal parameters are described herein above.

According to some exemplary embodiments, the responsiveness of the subject to the therapeutic agent/therapy is determined at block 108. The responsiveness is determined based on the valued obtained at block 106.

The responsiveness may be determined, for example, by comparing the at least one respiration parameter value with a reference value, wherein a difference between the two is indicative of responsiveness.

The comparison between the valued can be made utilizing techniques such as reference limits, discrimination limits, or risk defining thresholds to define cutoff points and abnormal values.

In one embodiment, the reference value is the value derived from the subject prior to administration of the therapeutic agent/therapy. The reference value may be obtained immediately prior to administration of the therapeutic agent or may be obtained on a different occasion.

In some embodiments of the invention, when the value of the respiratory parameter of the tested subject is sufficiently dissimilar (e.g. statistically significantly similar) to a reference value derived from the subject prior to administration of the medication/therapy, it is indicative that the treatment is efficacious. When the value of the respiratory parameter of the tested subject is similar (e.g. statistically significantly similar) to a reference value derived from the subject (prior to administration of the medication/therapy), it is indicative that the treatment is not efficacious.

In one embodiment, the reference value is the value of a control value which is derived from a different subject (or group of subjects) suffering from ADHD. When the value of the respiratory parameter of the treated subject is sufficiently similar (e.g. statistically significantly similar) to a reference value derived from an ADHD subject (or group of subjects), it is indicative that the treatment is not efficacious. When the value of the respiratory parameter of the treated subject is sufficiently dissimilar (e.g. statistically significantly dissimilar) to a reference value derived from an ADHD subject (or group of subjects), it is indicative that the medication/therapy is efficacious. In one example, the group of reference subjects is selected based on one or more of gender, BMI, age and clinical/diagnostic information (such as questionnaire data). Optionally or additionally, the reference group is selected based on similarity of nasal parameters. Optionally, the groups are selected based on a post-hoc analysis showing which groups of subjects best (or good enough) predict. In one example, after generating an ADHD classifier based on a set of, for example, 1000 subjects, a series of classifiers are generated using various subsets of the subjects and the subset which provides a best prediction is determined to be a good reference group. This subset may also be used to define similarity of nasal parameters. Other methods of optimization and subset selection may be used as well.

In one embodiment, the reference value is the value of a control value that is derived from a subject or group of subjects not suffering from ADHD. When the value of the respiratory parameter of the treated subject is sufficiently similar (e.g. statistically significantly similar) to a reference value derived from a non-ADHD subject (or group of subjects), it is indicative that the treatment is efficacious. When the value of the respiratory parameter of the treated subject is sufficiently dissimilar (e.g. statistically significantly dissimilar) to a reference value derived from a non-ADHD subject (or group of subjects), it is indicative that the medication/therapy is non-efficacious.

FIG. 8B is a flow chart of a process for ongoing subject monitoring, according to some exemplary embodiments of the invention.

At 802 reference state data is optionally collected, for example, to determine a starting ADHD status. Methods as described herein may be used.

At 804, information may be provided, for example, sensor information regarding the subject physiology or the environment. Optionally or additionally, a subject may be requested or may volunteer information, for example, mood, answers to various questions, ongoing or planned tasks, foods, other information regarding things which may affect ADHD status or measurements thereof (e.g., recent asthma attack), including taking of medication. UI 222 may be used for such data collection and memory 214 may be used for storing data. Control circuitry is optionally used to manage the process of FIG. 8B (and/or of other methods described herein).

The subject may also input desired ADHD status, for example, timing of a difficult class or other activity, so that system 200 can provide recommendations which increase subject attention when desired by the subject.

At 806, a change in ADHD state is monitored. Such monitoring may be, for example, ongoing, on a timed basis and/or in response to a subject's request or a trigger form an external system (e.g., an app on a subject's cellphone, an automated teaching system which detected reduced attention).

In some embodiments of the invention, the change is detected as a change in a score. In other embodiments, an absolute state is evaluated and then compared to a reference absolute state. It is noted that a relative change in status may be easier to detect than a change in absolute status. For example, a slight increase in ADHD score may still maintain the subject in a same ADHD status bucket, but is indicative, especially if it is part of a trend, of a change in actual ADHD state of the subject.

At block 808, in some embodiments of the invention, such small changes are optionally used to predict an upcoming significant change in ADHD state and/or a timing thereof. For example, system 200 may include as a reference an expected (e.g., based on measured or standard) change in ADHD score as a function of time and/or medication. Such data may be used to predict when, for this particular subject, a certain ADHD state will be reached and/or when an additional therapy (such as medication, optionally including a dose amount) would be useful to provide. In some embodiments of the invention, such calculations are made by system 200, for example at a remote processor 212 thereof.

Other comparisons may be provided at 808, for example, various activities, for example, as measured and/or input at 804 may be correlated with ADHD state.

In some embodiments of the invention, measurements, such as environmental measurements are collected so as to update a personal reaction dataset of the subject. Such a data set may be used for a calibration process and/or baseline correction and/or for more advanced diagnosis of the subject as suffering for a particular profile of ADHD activity.

At 810 one or more recommendation may be made to a subject, for example, to do an activity, stop and activity or take medication.

Alternatively or optionally, at 810 other data may be provided to a subject, for example, at the end of a day or at some other periodicity, the subject may be advised regarding the typical ebbs and flows of the ADHD state (e.g., with or without medication).

According to some exemplary embodiments, ADHD-status system, for example as described with reference to FIG. 9A is used to determine responsiveness of an ADHD subject to an agent or therapy and/or monitor ADHD status over a day or other time period.

According to some exemplary embodiments, a nasal respiration monitoring system receives the signals recorded at the measurements sites, and generates an indication with respect to the responsiveness of a subject to the agent/therapy, optionally in a form of index measurements. In some embodiments, the system presents the responsiveness indication to a user.

It is noted that for the methods of FIGS. 8A, 8B, 10 and 11, as well as other methods described herein, ADHD detection methods other than nasal parameters may be used (e.g., sensor 204 is replaced by a different sensor or input type) and the methods described herein applied on non-nasal information. Alternatively or optionally, additional sensors which also provided ADHD indication may be used, and the combined data from multiple sensors may be used to provide a potentially more accurate ADHD status. For example, accelerometers appear to have been used to generate ADHD indication and/or characterized certain movement patterns as being more typical of ADHD sufferers (e.g., Muñoz-Organero, M., Powell, L., Heller, B., Harpin, V., & Parker, J. (2018). Automatic Extraction and Detection of Characteristic Movement Patterns in Children with ADHD Based on a Convolutional Neural Network (CNN) and Acceleration Images. Sensors (Basel, Switzerland), 18(11). doi: doi(dot)org/10.3390/s18113924). In some cases, measurements using one sensor (and processing thereof) are used to trigger measurements using another sensor, for example, if the first measurements seem indicative of a change in ADHD status.

A particular potential advantage of nasal information is that breathing is a core function, as compared to, for example, movements. Another particular potential advantage of nasal measurements is that they may be less affected by movements of a subject (e.g., due to activity, such as computer or mobile device use). Another particular potential advantage of nasal measurements is that breathing may be more easily trained and/or controlled than fidgeting. Another potential advantage of nasal measurements is that they may be more difficult to fake and/or unintentionally control. Another potential advantage of nasal measurements is that measurement may be faster and/or include less variance, this may result from any of the advantages noted herein. Another potential advantage of nasal measurement is that they can be used for any sedentary activity and also during sleep (optionally being normalized to an indication of neural or other activation level, such as pulse or heart rate variability). Another potential advantage of nasal measurements is that the breathing is under direct neural control and what is measured is changes in the breathing due to such control, rather than what is effectively noise in intended movements. Another potential advantage for nasal measurements is that while the measured signal is simpler (and similar across people and activities), the number of parameters is not small and can be focused all on the same activity, potentially leading to better results when building a classifier or applying other machine learning methods.

According to some exemplary embodiments, a control circuitry optionally generates a confidence index. In some embodiments, the confidence index is calculated based on the analyzed signals and/or based on one or more indications stored in the memory of the system. In some embodiments, values of the confidence index indicate a degree of confidence of the calculated values of the responsiveness of the subject to the therapy and/or on a change in ADHD status.

Additionally, in some embodiments, the index of responsiveness is generated using one or more subject-related indication stored in a memory of the system and/or in the remote device. In some embodiments, the subject-related indication comprise indication regarding at least one of, clinical state of the subject, subject age, subject BMI, medical history of the subject and/or drug regime of the subject. UI 222 may be used to allow a user to input information regarding the tested therapy—e.g. dose, timing, regimen, number of times user has been exposed to the therapy.

According to some exemplary embodiments, the responsiveness index of the subject to the therapy are communicated to a user of the system, for example to the subject himself and/or to a professional. In some embodiments, the indications are delivered using a user interface, for example user interface 222 or using a display operationally connected to the user interface 222.

### Exemplary Process for Monitoring the Progression of ADHD of a Subject

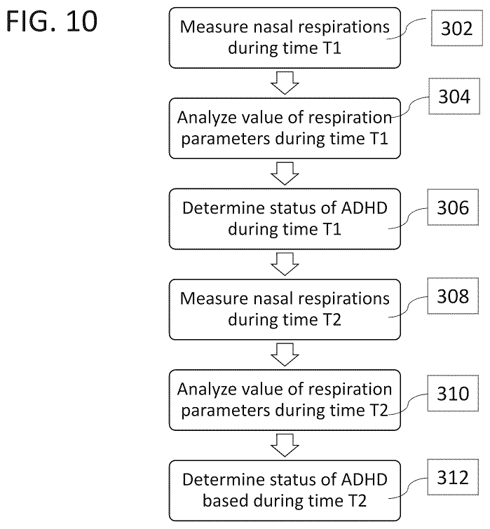

Reference is now made to FIG. 10, depicting a process for monitoring progression of ADHD subject over time, according to some exemplary embodiments of the invention. This process is particularly useful for generating treatment regimens which can be personalized according to the status of the ADHD of the subject at a particular point in time.

Nasal respirations are measured at block 302 at a particular point in time. The measurements are carried out for a length of time suitable to make an assessment of the subject regarding ADHD status. In one embodiment, the measurements are carried out for at least one minute, two minutes, five minutes, 10 minutes, 20 minutes, 30 minutes, one hour or longer. The measurements may be continuous or non-continuous. The measurement device may be placed at a position suitable for recording nasal respirations.

In one embodiment, measurement at block 302 is made at the start of a treatment or after a predetermined amount of time following a treatment (allowing sufficient time for the treatment to bring about its effect). This may be referred to as a baseline measurement.

Values of nasal respiratory parameters are analyzed at block 304. Exemplary parameters of nasal parameters are described herein.

According to some exemplary embodiments, the status of the subject at time T1 is determined at block 306. The status is determined based on the valued obtained at block 304.

The status may be determined by comparing the at least one respiration parameter value with a reference value, wherein a difference between the two is indicative of responsiveness, as further described herein above.

The comparison between the valued can be made utilizing techniques such as reference limits, discrimination limits, or risk defining thresholds to define cutoff points and abnormal values.

A second measurement continues at block 308. The second measurement may be carried out at a predetermined time (e.g. at least one hour after measurement at block 302, at least two hours after measurement at block 302, at least three hours after measurement at block 302, at least four hours after measurement at block 302, at least five hours after measurement at block 302, at least two hours after measurement at block 302, at least six hours after measurement at block 302, or for longer or intermediate or shorter times). The second measurement may be carried out in order to determine if the effect of a therapeutic agent useful for treating ADHD provided during or prior to T1 has worn off, or diminished. It will be appreciated that the measurement may be a continuous measurement from time T1 until time T2 (and optionally longer), or may be a separate measurement, wherein a first measurement is carried out at 302 and then terminated and a subsequent measurement is carried out at 308. It will be appreciated that if the measurement is continuous, the device is typically not removed during the time between T1 and T2. If a separate measurement is made, the device may be removed following measurement at T1 and replaced at the site of measurement at time T2.

Values of nasal respiratory parameters are analyzed at block 310. Exemplary parameters of nasal parameters are described herein.

According to some exemplary embodiments, the status of the subject at time T2 is determined at block 312. The status is determined based on the valued obtained at block 310.

The status may be determined by comparing the at least one respiration parameter value with a reference value, wherein a difference between the two is indicative of responsiveness, as further described herein above.

It will be appreciated that the measurements may be carried out at a plurality of times during the day, for example at least twice a day, at least three times a day, at least four times a day, at set times and/or in response to various triggers (manual and/or automatic).

Optionally, the process may continue by comparing the ADHD status ascertained at block 306 with the ADHD status ascertained at block 314. In this way, it is possible to monitor the progression of the ADHD from time T1 until time T2.

In some embodiments of the invention, the overall effect of a therapy, for example, biofeedback, is assessed by T1 and T2 covering a period of, for example, 1-5 days, weeks or months. More than two measurements may be made as well. Changes over such longer periods of time can indicate a general disorder burden on the subject.

According to some exemplary embodiments, a system, for example system 200 shown in FIG. 9A and as described herein, is used to determine progression of ADHD of a subject over time. In some embodiments, nasal respiration is monitored by monitoring nasal airflow of at least one nostril of a subject using a device comprising a sensor 204 (e.g. pressure sensor).

According to some exemplary embodiments, a nasal respiration monitoring system receives the signals recorded at the measurements sites, and generates an indication with respect to the ADHD of the subject at particular time points (e.g. during the course of a day), optionally in a form of index measurements. In some embodiments, the system presents the progression of the ADHD to a user.

As noted, in some embodiments, the index of ADHD status is generated using one or more subject-related indication stored in a memory of the system and/or in the remote device. In some embodiments, the subject-related indication comprise indication regarding at least one of, clinical state of the subject, subject age, subject BMI, medical history of the subject and/or drug regime of the subject. The system may comprise an input circuitry connected to the memory which allows a user to input information regarding a tested therapy—e.g. dose, timing, regimen, number of times user has been exposed to the therapy.

According to some exemplary embodiments, the responsiveness index of the subject to the therapy or otherwise changes over time are communicated to a user of the system, for example to the subject himself and/or to a professional. In some embodiments, the indications are delivered using a user interface, for example user interface 222 or using a display operationally connected to the user interface 222.

According to some exemplary embodiments, the control circuitry is configured to process and/or to generate the nasal flow and/or ADHD status indication, using one or more algorithms stored in the remote device. In some embodiments, the control circuitry transmits the signals received from the sensors or indications thereof to the remote device, and received processed signals or the status indication from the remote device. Optionally the processing and/or the generation of the status indication is performed in the remote device 212 using one or more algorithms stored in the remote device 212.

### Exemplary Process for Altering ADHD Status of a Subject by Providing Feedback to the Subject Regarding Nasal Respirations

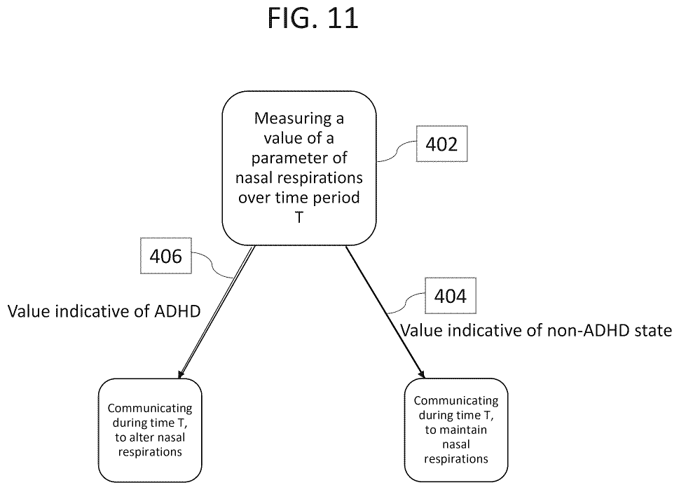

Reference is now made to FIG. 11, depicting a process for altering ADHD status of a subject by providing feedback to the subject regarding nasal respirations, according to some exemplary embodiments of the invention. This process may be used for improving management of ADHD and typically comprises continuous measuring and real-time analysis.

Nasal respirations are measured over a time period T. The measurements are carried out for a length of time suitable to make an assessment of the subject regarding ADHD status. Values of nasal respiratory parameters are analyzed at block 402. Exemplary parameters of nasal parameters are described herein above. According to some exemplary embodiments, the status of the subject at time T1 is determined based on the valued obtained.

The status may be determined by comparing the at least one respiration parameter value with a reference value, wherein a difference between the two is indicative of responsiveness, as further described herein above.

The comparison between the valued can be made utilizing techniques such as reference limits, discrimination limits, or risk defining thresholds to define cutoff points and abnormal values. It is noted in this and other embodiments, measurement need not stop when ADHD status is calculated. For example, a moving window of measurements may be used to calculate a continuously changing value for ADHD status.

The status is optionally communicated to the user at blocks 404 and 406. In one embodiment, the status indicates that the nasal respirations of the subject are indicative of ADHD (block 406). The subject then alters or modifies his nasal respirations (e.g. longer breaths, shorter breaths, breathing only through nose, breathing only orally etc.) in an attempt to modify his nasal respiration profile such that his ADHD status improves (i.e. more closely resembles a non-ADHD subject). Values of nasal respiratory parameters are re-analyzed and communicated back to the subject. Optionally, the subject learns through this process, which nasal respiratory patterns are helpful in managing ADHD and which nasal respiratory patterns are not helpful.

In one embodiment, the status indicates that the nasal respirations of the subject are indicative of non-ADHD (block 404). The subject then continues his nasal respirations in a similar pattern in an attempt to retain the positive nasal respiration profile such that his ADHD status remains negative (i.e. more closely resembles a non-ADHD subject). Values of nasal respiratory parameters are re-analyzed and communicated back to the subject. The subject learns through this process, which nasal respiratory patterns are helpful in managing ADHD and which nasal respiratory patterns are not helpful.

According to some exemplary embodiments, a nasal respiration monitoring system receives the signals recorded at measurements sites, and generates an indication with respect to the ADHD of the subject at particular time points, optionally in a form of index measurements. In some embodiments, the system presents the progression of the ADHD to a user in real-time allowing the user to adapt his nasal respiratory patterns so as to obtain a more positive indication of his ADHD.

In some embodiments of the invention, the method includes training a subject in applying “correct” breathing patterns, for example, breathing with a lower exhalation volume and/or velocity for exhalation and/or inhalation (e.g., as shown in the experimental results). Optionally, an existing breathing training system is used, programmed with breathing patterns as described herein Alternatively or optionally, it is noted that different breathing patterns may be effective for different users. Methods, such as in FIGS. 8A, 8B and 10 may be used to guide a subject to try different breathing patterns (optionally confirming they are followed by measurement of nasal breathing parameters in response to the guidance) and then check ADHD status, for example, using nasal parameters during a time when the subject is instructed to “breath regularly” or using some other ADHD indicator, such as a TOVA test or other indications as known in the art.

In some embodiments of the invention, the system (e.g., system 200 of FIG. 9A) does not show the user feedback re ADHD status, instead simply providing guidance how to breath (e.g., with therapeutic breathing guidance provided if needed and/or if not being followed in spite of guidance), based on subject need. In one example, where the breathing pattern comprises reducing peak velocity, an indicator, for example, visual or audio or possibly vibrational, is provided to the subject to indicate a breathing velocity and/or if the velocity is too low or too high. Such an indicator may be part of system 200 or may be, for example, part of a UI 222, possibly part of a subject's cellular telephone. In one example, such a vibration feedback is provided on a same component as is located near a nostril to measure airflow parameters.

In some embodiments of the invention, the subject is not trained on certain parameters, such as nasal cycling and the measurement of such parameter(s) is used as an independent indication as to whether the breathing exercise is affecting the subject's actual ADHD-status. If the nasal cycling rate goes down, this may be an indication that ADHD status is actually changing with the breathing.

### Device for Measuring Nasal Respirations

In some embodiments, the device is a sensor, for measurement of respiration e.g. of nasal airflow of the subject. In some embodiments, the sensor/s include a spirometer. In some embodiments, airflow sensor/s are fluidly connected to a cannula or probe which is placed within the subject's nasal passageway. In some embodiments of the invention, an airflow sensing system such as sold by sniff logic LTD of Tel-Aviv, Israel, may be used.

In some embodiments of the invention, measurements of nasal airflow are taken at 6 Hz. Higher or lower frequencies may be used.

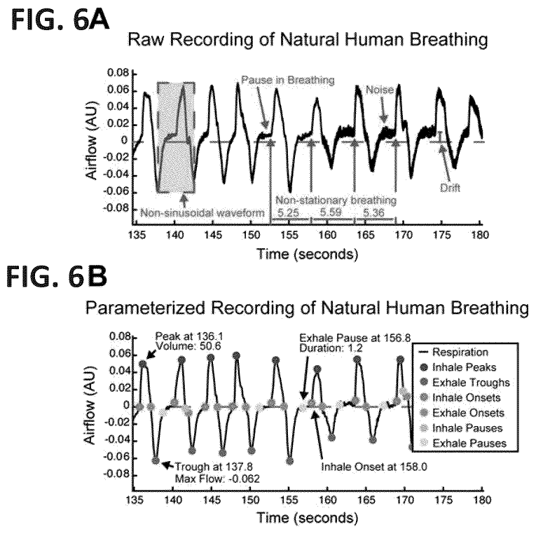

In some embodiments, the device comprises at least two independent sensors, one for measuring nasal airflow in the right nostril and one for measuring nasal airflow in the left nostril. The device may comprise a left nostril pressure probe which is configured to be inserted into a left nostril of a subject, and includes a left-nostril-pressure tube that is configured to transmit a left-nostril pressure wave from the left nostril to the left-nostril pressure sensor; a right-nostril pressure probe, which is configured to be inserted into a right nostril of the subject, and includes a right-nostril-pressure tube that is configured to transmit a right-nostril pressure wave from the right nostril to the right-nostril pressure sensor. An exemplary signal which can be recorded using such a device is shown in FIG. 6A. The separate nostril sensors may be used, for example, for noise reduction, for example by averaging or selecting a best signal.

In some embodiments of the invention, the device further includes a pressure probe for analyzing oral pressure and/or oral respiratory patterns.

Alternatively or additionally, in some embodiments, temperature of nasal airflow is measured, for example by thermistor/s placed within the nasal air flow (and e.g. not in contact with the skin).

Alternatively or additionally, in some embodiments, a pneumotachometer is used to measure differential pressure of the nasal air flow. Where, in some embodiments, the differential pressure is converted into a voltage signal using a spirometer.

In an exemplary embodiment, nasal airflow is measured using a nasal cannula (e.g. 1103, Teleflex medical) linked directly to a spirometer (e.g. ML141, ADInstruments, H₂O resolution=15.6 μV). Where the spirometer, in some embodiments, converts airflow into a voltage signal. In an exemplary embodiment, the airflow voltage signal is amplified by an instrumentation amplifier (e.g. PowerLab 16SP Monitoring System, ADInstruments).

In some embodiments, data is collected by sampling the airflow voltage signal. In some embodiments, the airflow signal is sampled at 100-10,000 Hz, or 500-200 Hz, or at about 1000 Hz, or lower or higher or intermediate ranges or sampling rates. In an exemplary embodiment, sampling is at 1000 Hz.

In an exemplary embodiment, sampling is using LabChart software (ADInstruments). An exemplary device that can be used to measure nasal respirations is disclosed in U.S. application Ser. No. 17/380,348 now US patent publication 2023/0028914A1, the contents of which are incorporated herein by reference.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Nasal Respiratory Parameters can be Used to Diagnose ADHD

In all, 34 participants with ADHD (17M, 26.7±3.1YO) and 37 controls (17M, 25.7±4.1YO) were included in the study. Participants wore a halter for 24 hours continuously.

### Results

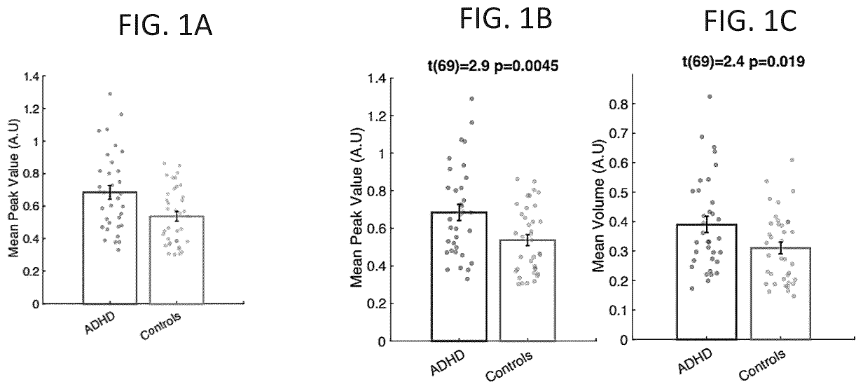

Inhalations in ADHD were significantly larger (higher peak and volume), mean peak value ADHD=0.69±0.28 arbitrary units (AU), mean control=0.52±0.18, t(69)=3.07 p=0.003, mean inhales volume ADHD=0.41±0.18 AU mean control=0.31±0.12, t(69)=2.63 p=0.01) (FIG. 1A).

ADHD inhalations and exhalations have significantly higher peak during wake time compared to control (FIGS. 1B and 1C, respectively). The difference between ADHD and control was maintained during cognitive processing (study time), as shown in FIGS. 1D and 1E, respectively.

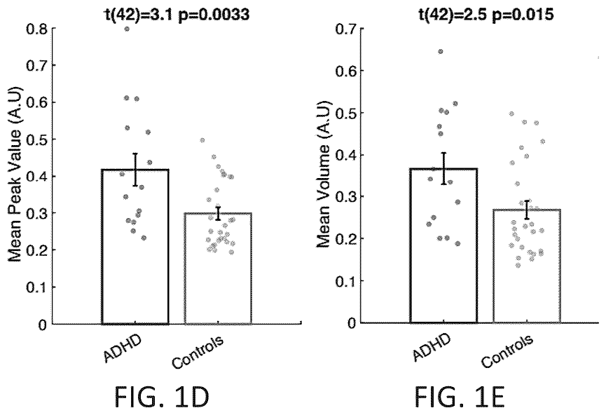

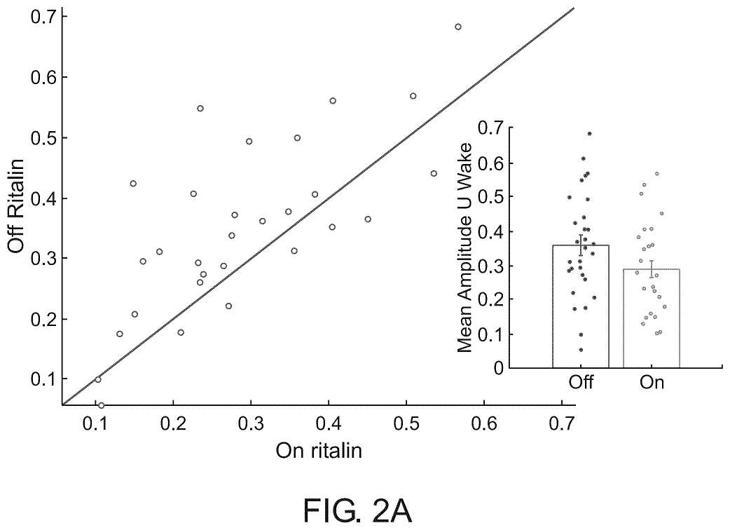

Moreover, 28 of the ADHD participants were measured twice, once “ON” and once “OFF” MPH (Ritalin). A significant shift in the Laterality amplitude (LA) of the Nasal Cycle (i.e., the extent of difference across nostrils, for example as in Kahana-Zweig et al., PLOS One. 2016, 11(10):e0162918.) was observed, that was reduced on MPH (LA “ON”=0.3±0.14 AU, LA “OFF”=0.35±0.15, t(27)=2.8, p=0.008) (FIG. 2A). This may be used as part of an ADHD classifier, in accordance with some embodiments of the invention.

Finally, a shift in dominant nostril, from Left off MPH to Right on MPH (LI “ON”=0.04±0.2, LI “OFF”=−0.03±0.2, t(27)=2.4, p=0.02) was observed.

FIG. 2B shows that Ritalin reduces inhalation and exhalation amplitude, which may be used as part of an ADHD classifier, in accordance with some embodiments of the invention.

### Example 2

### Nasal Respiratory Parameters can be Used to Train a Neural Network Classifier in Order to Diagnose Subjects with ADHD

90 subjects (30 ADHD), filled ASRS questionnaire and nasal breathing was recorded for 24 hours using a nasal holter (e.g., as disclosed in U.S. patent application Ser. No. 17/380,348, now published as US patent publication 2023/0028914A1).

The raw data were normalized and divided to time series of 5-minutes. Then the data was analyzed using the 25 respiratory parameters mentioned above. Next, the artificial neural network classifier described above was used. Training data included 95% (n−1) of the ADHD and on balanced control group, and the testing consist 1 control and 1 ADHD.

### Results

The classifier succeeded to diagnose at general accuracy of 70%. (chance is 50%). Importantly, even only one hour of recording was sufficient for this accuracy level.

### Example 3

### Nasal Respiratory Parameters can be Used to Distinguish Between a Ritalin-Treated ADHD Subject and Non-Treated Subject in a Short Time Span

Nasal respiration was measured in 30 ADHD subjects for 24h twice-on and off treatment, using the above classifier.

### Results

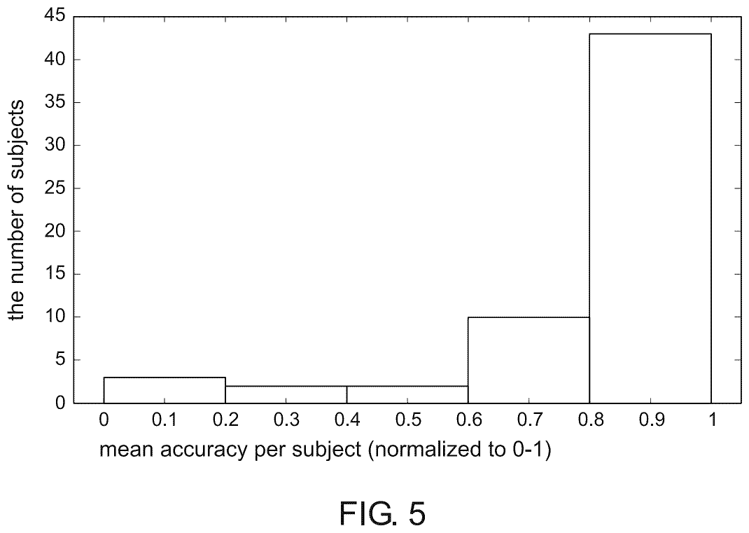

As shown in FIG. 5, nasal respiratory parameters can be used to distinguish between ritalin-treated/non-treated subjects.

5 minutes recording was found to be sufficient to detect if the subject is on or off Ritalin in 82.5±1.24% of events. For over half of the patients (53.33%), 5 minutes was sufficient to detect Ritalin status with a mean accuracy higher than 90%. It is noted that the curve is biased. In some embodiments of the invention, a subject is tested to see how reliable and accurate ADHD state testing is for them. This may be used, for example, to select which subjects use nasal monitoring for more accurate estimation of instantaneous ADHD state.

As noted above, as Ritalin wears off, an intermediate number may be used to provide a “Ritalin-score” or an “ADHD-score”, in accordance with some embodiments of the invention. A graph of Ritalin response may be collected per subject, optionally automatically, if a subject enters Ritalin dosage and time into system 200 and system 200 tracks the ADHD-score of the subject over time. In some embodiments of the invention, a new classifier is created with different ADHD states. Alternatively, the score assumes a linear effect of one or more parameters, such as the flow velocity. Optionally or additionally, population results are used to identify values that are less or more likely to indicate ADHD “on” and “off” states and these are used to indicate a lower or higher ADHD score according to the population statistics.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for assessing an ADHD status of a subject comprising:
receiving circuitry configured to receive a measurement signal including measurements of a plurality of nasal respirations from a sensor configured to sense a nasal respiration of a subject;
determining circuitry configured to determine values of one or more nasal respiration parameter from said measurement signal;
evaluation circuitry configured to evaluate said subject, based on said values of one or more respiration parameter to provide an indication of ADHD status of said subject.

2. A system according to claim 1, comprising at least one nasal flow sensor which provides said measurement signal.

3. The system of claim 1, wherein said plurality of nasal respirations comprises at least two consecutive nasal respirations of the subject.

4. The system of claim 1, wherein said measurement signal includes at least 10% of all breaths over a time period of at least five minutes.

5. The system of claim 1, wherein said at least one respiration parameter comprises a volume-associated parameter or a timing-associated parameter.

6. The system of claim 1, wherein said at least one respiration parameter comprises a timing-associated parameter.

7. The system of claim 6, wherein said timing-associated parameter comprises at least one of a duration of a nasal inhalation, a duration of a nasal exhalation, a time between two nasal inhalations, a duty cycle and a time between two nasal exhalations.

8. The system of claim 1, wherein said at least one respiration parameter comprises at least one of a breathing rate, an inter-breath interval, an inhale volume, an exhale volume, a tidal volume, a minute ventilation and a duty cycle.

9. The system of claim 8, wherein said at least one parameter comprises inhalation and/or exhalation velocity and/or duration.

10. The system of claim 1, wherein said evaluating comprises evaluating one or both of a coefficient of variation of said parameter over a predetermined time period and a peak value of said parameter over a predetermined time period.

11. (canceled)

12. The system of claim 1, wherein said evaluation circuitry is configured to monitor at least one parameter of a nasal respiratory waveform of the subject.

13. The system of claim 1, wherein said evaluation circuitry includes a classifier trained to classify nasal breathing data into ADHD status.

14. The system of claim 1, wherein said ADHD status comprises an ADHD sub-classification or an ADHD severity score.

15. (canceled)

16. The system of claim 1, wherein said evaluation circuitry is configured to compare said value of said at least one respiration parameter with a reference value, wherein a difference between said value of at least one respiration parameter of said subject and said reference value is indicative of a status of ADHD.

17. The system of claim 1, comprising memory storing therein at least one reference value personalized for the subject.

18. (canceled)

19. The system of claim 1, wherein said indication comprises:
(i) a diagnosis of ADHD;
(ii) a change in status of ADHD;
(iii) recommendation to take medication or apply a therapy; and/or
(iv) an indication regarding an effectiveness therapy.

20-27. (canceled)

28. A method of determining an Attention Deficit Hyperactivity Disorder (ADHD) status of a subject comprising:
measuring a plurality of nasal respirations of the subject, and
determining a value of at least one respiration parameter of said plurality of nasal respirations,
generating an indication of the ADHD status of the subject based on said value of said at least one respiration parameter.

29-30. (canceled)

31. A method according to claim 28, wherein said measuring comprises measuring in a timed relationship to taking of ADHD-related medication by the subject.

32-40. (canceled)

41. A method of determining whether a therapy or therapeutic agent is effective in managing ADHD in a subject comprising:
applying the therapy to the subject or administering the therapeutic agent to the subject; and
determining a status of ADHD in a time window shorter than 15 minutes, wherein the indication of ADHD is a responsiveness to the therapy or therapeutic agent, wherein said applying or administering is effected prior to or during the measuring.

42. A method according to claim 41, wherein said applying comprises indicating such applying or administering to a system used to measure the ADHD status.

43-50. (canceled)
